(19) United States
(12) Patent Application Publication
(10) Pub. No.: US 2008/0083348 A1
(43) Pub. Date: Apr. 10, 2008

(54) Dual-chamber package for preparing alginate impression paste

(75) Inventors: Eric Kuo, Foster City, CA (US); Ken Wu, San Francisco, CA (US); Robert E. Tricca, Danville, CA (US)

Correspondence Address: KLEIN, O'NEILL & SINGH, LLP, 43 CORPORATE PARK, SUITE 204, IRVINE, CA, 92606, US

(73) Assignee: ALIGN TECHNOLOGY, INC., Santa Clara, CA (US)

(21) Appl. No.: 11/343,917

(22) Filed: Jan. 31, 2006

(51) Int. Cl. C09K 3/00; B65D 25/08; B65D 69/00; A61B 19/02

(52) U.S. Cl. 106/35; 206/219; 206/568; 206/63.5

(57) ABSTRACT

A dual-chamber package for the constituent materials for preparing an alginate dental impression paste includes a first chamber containing a pre-measured volume of water, and a second chamber, attached to and isolated from, the first chamber, and containing a pre-measured quantity of alginate powder, wherein the quantity of powder and the volume of water are in a pre-determined ratio for making a dental impression paste when mixed together. The powder and the water may be mixed outside of the package by dispensing the water and the powder respectively from the first and second chambers. Alternatively, the powder and water may be mixed within the second chamber by breaching a barrier between the first and second chambers to allow water to flow from the first chamber into the second chamber, after which the alginate dental impression paste is dispensed from the second chamber.

### CROSS-REFERENCE TO RELATED APPLICATION

Not Applicable

### FEDERALLY-SPONSORED RESEARCH OR DEVELOPMENT

Not Applicable

### BACKGROUND OF THE INVENTION

This invention relates to the field of dentistry. More specifically, it relates to a device and a method for preparing the alginate material used to make dental impressions.

In dentistry, and particularly in the field of orthodontics, it is frequently necessary to create a stone model that is an accurate replica of the hard and soft tissues (i.e, teeth and gums) of a patient's dental arch. The first step in making the stone model is to make a dental impression, using an impression material. Typical impression materials include alginate (irreversible hydrocolloid), agar (reversible hydrocolloid), polysulfide, polyether, and polyvinylsiloxane (PVS). Impression materials are generally transferred to the patient's mouth in an impression tray. The tray is required because the materials are initially fluid and require support. Once the tray is positioned on the patient's dental arch, the materials undergo setting by either a chemical or physical process. After setting, the impression is removed from the patient's mouth, and a stone model is prepared, using a dental gypsum material that is initially fluid and that can be poured into the impression, where it hardens to form a rigid replica of the dental arch.

Alginate dental impression material was developed in the later 1930's and is the most widely-used dental impression material. The alginate impression material is typically prepared by the dentist by mixing, in a suitable vessel, a predetermined quantity of dry alginate powder, taken from a bulk container, with a measured volume of water. The dentist depends on personal experience to mix the powder and water in the correct proportions or ratios to make a paste with the proper consistency. While small deviations from the ideal proportions are tolerable, larger deviations can have deleterious effects on the quality of the impressions and ultimately on the accuracy of the molds made therefrom.

It would therefore be advantageous to provide a packaging device in which pre-measured quantities of alginate powder and water are stored in the correct ratio. It would be of further advantage to provide such a packaging device from which the water and powder can be easily dispensed when they are to be mixed, and yet which keeps these two substances completely isolated from each other until dispensing occurs.

### SUMMARY OF THE INVENTION

Broadly, the present invention, in one aspect, is a dual-chamber package for the constituent materials for preparing an alginate dental impression paste, comprising a first chamber containing a pre-measured volume of water and a second chamber, attached to the first chamber and isolated therefrom, and containing a pre-measured quantity of alginate powder, wherein the quantity of powder and the volume of water are in a pre-determined ratio for the formation of an alginate dental impression paste when mixed together.

In another aspect, the present invention is a method of preparing an alginate impression paste, comprising the steps of (a) providing a package having first and second chambers attached to, but isolated from, each other, with a pre-measured volume of water in the first chamber and a pre-measured quantity of alginate powder in the second chamber, wherein the quantity of powder and the volume of water are in a pre-determined ratio for the formation of an alginate dental impression paste when mixed together; and (b) mixing the powder and the water to form an alginate dental impression paste. In one embodiment of the method, the mixing step is performed outside of the package and is preceded by a step of dispensing the water and the powder respectively from the first and second chambers. In another embodiment of the method, the mixing step is performed within the second chamber, and is preceded by a step of breaching a barrier between the first and second chambers to allow water to flow from the first chamber into the second chamber. In this second embodiment, the mixing step is followed by a step of dispensing, from the second chamber, an alginate dental impression paste formed in the second chamber during the mixing step.

A first specific embodiment of a dual-chamber package in accordance with the invention comprises a laminated package or pouch defining a first or water chamber and a second or powder chamber, wherein the first and second chambers are adjacent to each other, separated by a fluid-impermeable barrier. The package includes at least one tear-off portion adjacent the first and second chambers for opening the first chamber to dispense the water therefrom and the second chamber to dispense the powder therefrom. The first and second chambers may be arranged in parallel, and the first and second tear-off portions may be on opposite sides of the package, near a dispensing end thereof, adjacent the respective chambers, or they may be spaced apart from each other on the dispensing end of the package, adjacent the respective chambers. Alternatively, the first and second chambers may be arranged end-to-end, with one end of the package being a first dispensing end for the first chamber, and the other end of the package being a second dispensing end for the second chamber. A first tear-off portion is provided at the first end, and a second tear-off portion is provided at the second end, whereby the first and second chambers can respectively be opened to dispense the contents therefrom for mixing to form an alginate dental impression paste.

A second embodiment of a dual-chamber package in accordance with the invention, comprises a laminated package or pouch defining a first or water chamber and a second or powder chamber, wherein the first and second chambers are adjacent to each other, separated by a fluid-impermeable barrier having a frangible portion. The first chamber contains a pre-measured volume of water, and the second chamber contains a pre-measured quantity of alginate powder. The total volume of the powder chamber is sufficient to allow a thorough mixing of the water and the powder therein to create an alginate dental impression paste when the frangible portion of the barrier is breached or ruptured, allowing water to flow therethrough from the water chamber to the powder chamber. The pouch or package includes a tear-off portion adjacent the powder chamber, whereby a portion of the package can be removed to create an opening through which the paste formed in the powder chamber can be extruded (as by squeezing the powder chamber). Furthermore, the contents can be sealed and packaged under vacuum, such that mixing can performed with few or no air bubbles.

As will be more fully appreciated from the detailed description that follows, the present invention provides the dentist with the means for accurately mixing water and alginate powder in the proper ratio for making a dental impression paste with the optimum consistency for producing an accurate impression. Guess-work in this process removed, and accurate mixing is achieved with a convenient, easy-to-use package having pre-measured contents. Furthermore, the dual chamber package of the present invention is economical to manufacture, and, delivered in quantity, may replace bulk containers of alginate powder that can become contaminated by moisture and other environmental contaminants through repeated opening and closing.

### BRIEF DESCRIPTION OF THE DRAWINGS

FIG. 1 is plan view of a dual-chamber package in accordance with a first embodiment of the present invention;

FIG. 2 is an elevational view taken from the left side of FIG. 1;

FIG. 3 is a longitudinal cross-sectional view taken along line 3-3 of FIG. 2;

FIG. 4 is a lateral cross-sectional view taken along line 4-4 of FIG. 2;

FIG. 5 is a partial cross-sectional view, similar to that of FIG. 3, showing the removal of a tear-off portion of the package;

FIG. 6 is a longitudinal cross-sectional view of a dual-chamber package in accordance with a first variant of the first embodiment of the invention;

FIG. 7 is a partial cross-sectional view, similar to that of FIG. 6, showing the removal of the tear-off portions of the package;

FIG. 8 is a longitudinal cross-sectional view of a dual-chamber package in accordance with a second variant of the first embodiment;

FIG. 9 is a plan view of a dual-chamber package in accordance with a second embodiment of the invention;

FIG. 10 is a cross-sectional view taken along line 10-10 of FIG. 9;

FIG. 11 is a cross-sectional view taken along line 11-11 of FIG. 9, showing the barrier between the two chambers of the package before the barrier has been breached;

FIG. 12 is a cross-sectional view, similar to that of FIG. 11, showing the barrier after it has been breached; and

FIG. 14 is a partial cross-sectional view, showing the mixing of the water and the alginate powder in the first chamber after the barrier between the two chambers has been breached.

### DETAILED DESCRIPTION OF THE INVENTION

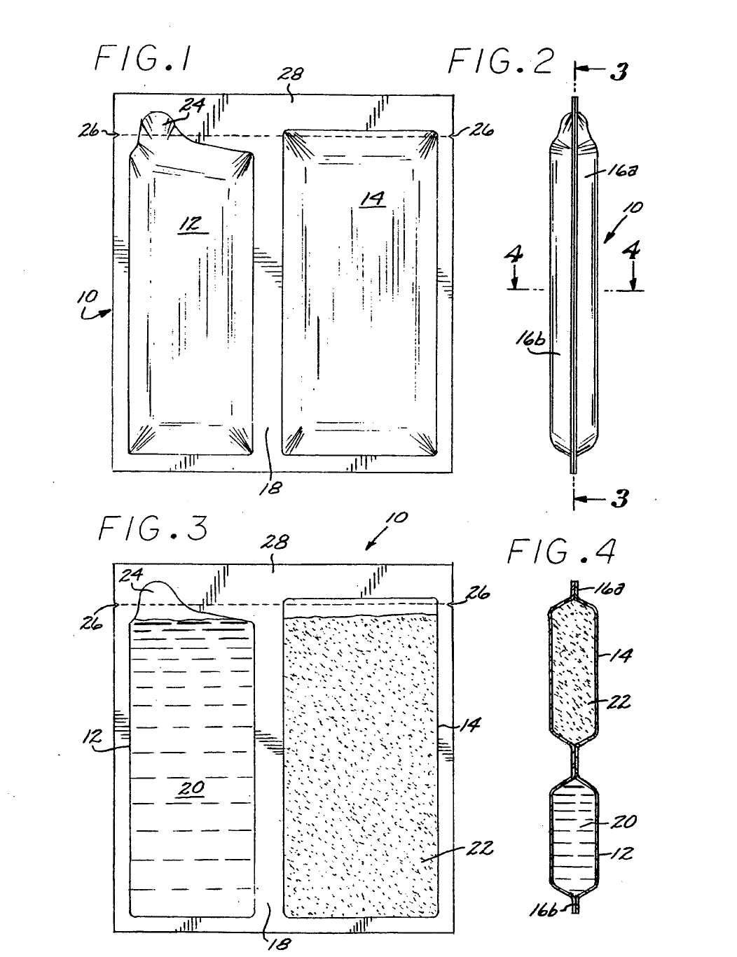

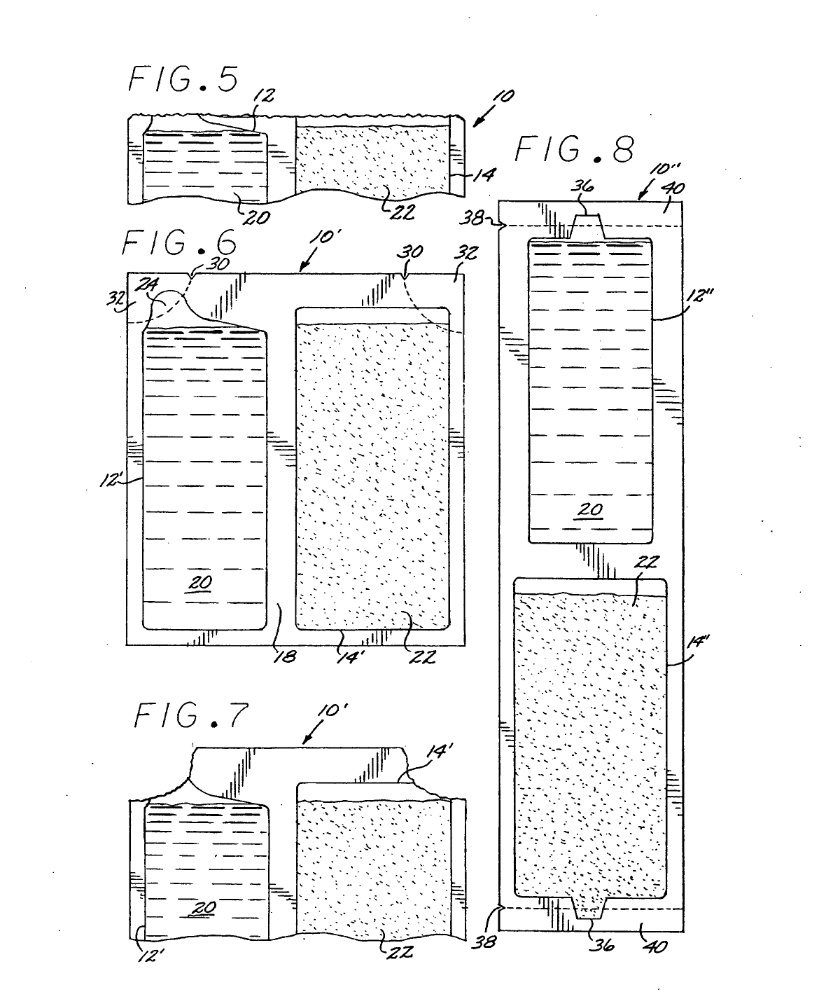

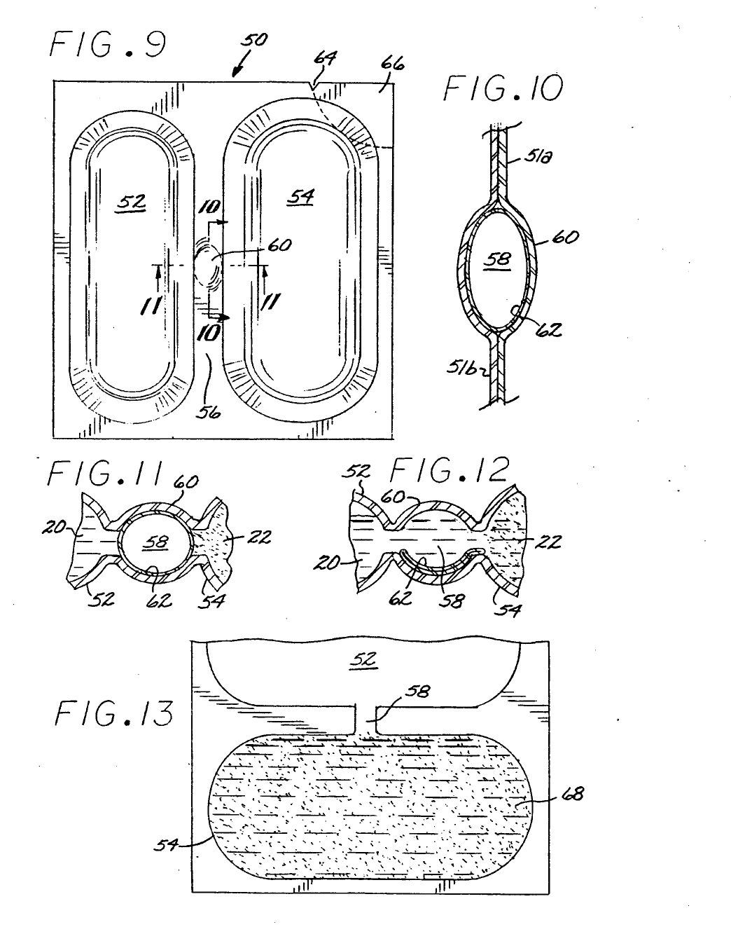

Referring first to FIGS. 1-5, a dual-chamber package 10, in accordance with a first embodiment of the present invention is shown. The package 10 is formed as a pouch having a first chamber 12 and a second chamber 14. The package 10 comprises two sheets 16a, 16b, preferably of a plastic/aluminum laminate material (see FIG. 2), each of which is formed with a pair of blisters defining either the front or back halves of the chambers 12, 14. The sheets 16a, 16b are preferably of a laminated sheet material that is gas and liquid impervious, with a high puncture resistance. One such material comprises a first layer of 48 gauge polyethylene terephthalate (PET), having a thickness of 0.00048 inches (0.012 mm); a second layer of low density polyethylene (LDPE), having a thickness of 0.00075 inches (0.019 mm); a third layer pf aluminum foil, having a thickness of 0.00028 inches (0.007 mm); a fourth performance tie (adhesive) layer, having a thickness of 0.00075 inches (0.019 mm); and a fifth layer of linear low density polyethylene film (LLDPEF), having a thickness of 0.00150 inches (0.038 mm). The first layer may be covered by a suitable ink layer, having a thickness of 0.00020 inches (0.005 mm). A suitable laminate material is marketed by Sonic Packaging Industries, of Westwood, N.J., under the trade name “Prime #2073”. The two sheets 16a, 16b are sealed to each other around the edges, and along a strip separating the chambers 12, 14 and forming a fluid-tight barrier 18 between them. The sealing may be by any suitable technique, preferably by means such as heat-sealing or ultrasonic sealing, and either with or without vacuum packaging.

As shown in FIGS. 3 and 4, the first chamber 12 contains a pre-measured volume of water 20 (preferably either USP or sterile), and the second chamber 14 contains a pre-measured quantity of alginate powder 22. In a specific preferred embodiment, the first chamber 12 is pre-filled with 40 ml of water, and the second chamber 14 is pre-filled with 18 g of alginate powder. These quantities are selected so that the water 20 and powder 22 are mixed in the proper ratios for optimal consistency of the alginate dental impression paste when the contents of the chambers 12, 14 are emptied into a mixing vessel (not shown). These quantities are also selected so that a usable amount of dental impression paste is produced for either an upper or lower impression when the water 20 and powder 22 are properly mixed.

As best shown in FIGS. 1 and 3, the first chamber 12 is preferably formed with an upper extension 24 that functions as a pour spout when the package 10 is opened, as described below. In addition, a tear notch 26 is formed on each of the side edges of the package 10, near the upper edge thereof. The tear notches 26 define a tear-off portion 28 of the package 10, which intersects the spout extension 24 of the first chamber 12 and the uppermost part of the second chamber 14, so that when the tear-off portion 28 is removed (FIG. 5), the chambers 12 and 14 are open to allow the contents of the chambers to be dispensed therefrom for mixing.

FIGS. 6 and 7 illustrate a package 10′ that is a first variant of the package 10 described above. The package 10′ is substantially identical to the package 10, except that a pair of tear notches 30 is provided on the upper edge of the package 10′, instead of the side notches 26 of the package 10. Each of the notches 30 is adjacent one of the chambers 12′, 14′, so that a tear-off portion 32 is provided separately for each of the chambers 12′, 14′ at each of the upper corners of the package 10′. This arrangement allows the chambers 12′, 14′ to be opened separately, thereby permitting the water to be dispensed before the powder is dispensed, which is the optimal sequence.

FIG. 8 illustrates a package 10″ that is a second variant of the package 10 described above. The package 10″ includes a first or water chamber 12″ and a second or powder chamber 14″ that are arranged end-to-end, with a fluid-tight barrier 34 between them. Each end of the package 10″ is a dispensing end for one of the chambers 12″, 14″, each of which has a dispensing spout 36 adjacent its respective dispensing end of the package 10″. A tear notch 38 is provided near each of the dispensing ends, whereby a tear-off portion 40 is formed across each dispensing end that intersects each of the dispensing spouts 36. This arrangement, like the first variant package 10′ described above, facilitates separate dispensing of the water 20 and the powder 22.

FIGS. 9-13 illustrate a dual-chamber package 50 in accordance with a second embodiment of the invention. The package 50 may be manufactured of the same or similar laminate material as that of the packages of the first embodiment described above, and like the packages of the first embodiment, it may be formed by two sheets 51a, 51b (FIG. 10) of that laminate material sealed to each other as described above. The package 50 includes a first chamber 52 containing a pre-measured volume of water 20, and a second chamber 54 containing a pre-measured quantity of alginate powder 22. The second chamber 54 is considerably larger than the first chamber 52, because, as will be explained below, the water 20 from the first chamber 52 is caused to flow into the second chamber 54 to be mixed with the powder 22 therein to form the dental impression paste.

The first and second chambers 52, 54 are separated by a fluid-impermeable barrier 56 that is interrupted by a passage or channel 58 that fluidly connects the chambers 52, 54. The channel 58 is defined within a small, central blister 60 in the package 50, and it is internally closed by a frangible element 62 that normally blocks flow between the chambers 52, 54. As shown, the frangible element 62 is formed as a bubble of rupturable plastic or polymer, but it may simply be a rupturable wall (not shown) spanning the passage or channel 58. A tear notch 64 is formed in the top edge of the package 50 near the second chamber 54, defining a tear-off portion 66 in one corner of the package 50.

In use, pressure is applied to the blister 60 to rupture the frangible element 62, thereby opening the channel 58, and allowing the water 20 to flow from the first chamber 52 into the second chamber 54, as shown in FIG. 12, when the first chamber 52 is squeezed. The water 20 mixes with the powder 22 in the second chamber 54, which is large enough in volume to permit a thorough mixing of the water and powder by agitation of the package 50. The result of the mixing is the formation of a dental impression paste 68 in the second chamber 54. The paste 68 can be dispensed from the second chamber 54 by tearing off the tear-off portion 66 to open the second chamber 54, and by then squeezing the second chamber 54 to extrude the paste 68 from the opening created by removing the tear-off portion 66.

From the foregoing, it will be appreciated that the present invention offers a convenient way for dentists to prepare, both accurately and repeatably, an alginate dental impression paste with the optimal proportions of water and alginate powder without guess-work. Furthermore, the present invention provides a single-use package for the alginate powder that avoids the possible contamination that may occur with bulk containers. The package according to the invention is economical to manufacture, easily shipped in large quantities, and easy to use.

Although preferred embodiments of the invention have been described herein, it will be appreciated that a number of variations and modifications will suggest themselves to those skilled in the pertinent arts. It will also be appreciated that the specific materials, configurations, and dimensions disclosed herein are exemplary only, and that other materials, configurations, and dimensions may be found suitable for particular applications. Such variations, modifications, and equivalents should be considered within the spirit and scope of the invention, as defined in the claims that follow.

What is claimed is:

1. A package for the constituent materials for making an alginate dental impression paste, comprising:
a first chamber containing a pre-measured volume of water; and
a second chamber, attached to the first chamber by a fluid-impermeable barrier, and containing a pre-measured quantity of alginate powder;
wherein the quantity of powder and the volume of water are in a pre-determined ratio for the formation of an alginate dental impression paste when mixed together.

2. The package of claim 1, wherein the package comprises:
a first sheet of material defining a first chamber front half and a second chamber front half; and
a second sheet of material defining a first chamber rear half and a second chamber rear half;
whereby the first and second sheets of material are joined together so that (a) the first chamber front and rear halves form the first chamber and the second chamber front and rear halves form the second chamber, and (b) the fluid-impermeable barrier is formed between the first and second chambers.

3. The package of claim 2, wherein each of the first and second sheets comprises an aluminum/plastic laminate.

4. The package of claim 1, wherein the package comprises first and second sheets of an aluminum/plastic laminate sealed to each other so as to form the first and second chambers and the barrier between them.

5. The package of claim 1, wherein the package includes a tear-off portion that is removable to open the first and second chambers to allow the dispensing of the contents thereof.

6. The package of claim 5, wherein the tear-off portion is a single tear-off portion that opens both the first and second chambers when removed.

7. The package of claim 5, wherein the tear-off portion includes a first tear-off portion that opens only the first chamber when removed, and a second tear-off portion that opens only the second chamber when removed.

8. The package of claim 5, wherein the first and second chambers are arranged in parallel and have respective dispensing ends at a first end of the package, the dispensing ends being openable by the removal of the tear-off portion.

9. The package of claim 7, wherein the first and second chambers are arranged end-to-end and have respective dispensing ends at opposite ends of the package, each of the dispensing ends being openable by the removal of one of the first and second tear-off portions.

10. The package of claim 1, wherein the barrier is interrupted by a passage fluidly connecting the first and second chambers, and wherein the passage is normally closed by a frangible closure that is openable by the application of pressure thereto, so that water from the first chamber can pass through the passage into the second chamber.

11. A method of preparing an alginate dental impression paste, comprising the steps of:
(a) providing a package having first and second chambers attached to, but isolated from, each other, with a pre-measured volume of water in the first chamber and a pre-measured quantity of alginate powder in the second chamber, wherein the quantity of powder and the volume of water are in a pre-determined ratio for the formation of an alginate dental impression paste when mixed together; and
(b) mixing the powder and the water to form an alginate dental impression paste.

12. The method of claim 11, wherein the mixing step is performed outside of the package and is preceded by a step of dispensing the water and the powder respectively from the first and second chambers.

13. The method of claim 11, wherein the mixing step is performed within the second chamber, and is preceded by a step of breaching a barrier between the first and second chambers to allow water to flow from the first chamber into the second chamber.

14. The method of claim 13, wherein the mixing step is followed by a step of dispensing, from the second chamber, the alginate dental impression paste formed in the second chamber during the mixing step.

15. A package for the constituent materials for making an alginate dental impression paste, comprising:
a first sheet of material defining a first chamber front half and a second chamber front half;
a second sheet of material defining a first chamber rear half and a second chamber rear half;
the first and second sheets of material being joined together so that (a) the first chamber front and rear halves form a first chamber and the second chamber front and rear halves form a second chamber, and (b) a fluid-impermeable barrier is formed between the first and second chambers;
a pre-measured volume of water contained in the first chamber; and
a pre-measured quantity of alginate powder contained in the second chamber;
wherein the quantity of powder and the volume of water are in a pre-determined ratio for the formation of an alginate dental impression paste when mixed together; and
wherein the package further includes a tear-off portion that is removable to open the first and second chambers to allow the dispensing of the contents thereof.

16. The package of claim 15, wherein each of the first and second sheets comprises an aluminum/plastic laminate.

17. The package of claim 15, wherein the tear-off portion is a single tear-off portion that opens both the first and second chambers when removed.

18. The package of claim 15, wherein the tear-off portion includes a first tear-off portion that opens only the first chamber when removed, and a second tear-off portion that opens only the second chamber when removed.

19. The package of claim 15, wherein the first and second chambers are arranged in parallel and have respective dispensing ends at a first end of the package, the dispensing ends being openable by the removal of the tear-off portion.

20. The package of claim 18, wherein the first and second chambers are arranged end-to-end and have respective dispensing ends at opposite ends of the package, each of the dispensing ends being openable by the removal of one of the first and second tear-off portions.

21. The package of claim 15, wherein the barrier is interrupted by a passage fluidly connecting the first and second chambers, and wherein the passage is normally closed by a frangible closure that is openable by the application of pressure thereto, so that water from the first chamber can pass through the passage into the second chamber.
